(19) United States
(12) Patent Application Publication
(10) Pub. No.: US 2020/0288983 A1
(43) Pub. Date: Sep. 17, 2020

(54) RESPIRATORY CORE BODY TEMPERATURE MEASUREMENT SYSTEMS AND METHODS

(71) Applicant: Masimo Corporation, Irvine, CA (US)

(72) Inventor: Valery G. Telfort, Irvine, CA (US)

(21) Appl. No.: 16/802,434

(22) Filed: Feb. 26, 2020

Related U.S. Application Data
(60) Provisional application No. 62/810,518, filed on Feb. 26, 2019.

(51) Int. Cl. A61B 5/01; A61B 5/0205; G01K 13/00; A61B 5/00; A61B 5/087

(52) U.S. Cl. CPC A61B 5/015; A61B 5/02055; A61B 5/0878; A61B 5/746; G01K 13/002

(57) ABSTRACT

Generally described, the present disclosure relates to measuring core body temperature through respiratory mechanisms. The disclosed techniques can use surface temperature, exhaled air, perfusion information, blood oxygen saturation, respiration rate, circadian rhythms, and the like to obtain an accurate reading of the body's core temperature. Example devices are disclosed for obtaining core temperature from exhaled air and useful mechanisms for presenting this information to a user are also disclosed, including user interfaces and alarm mechanisms. Stereo thermometry methods may also be used to estimate core body temperature. This information can be used to track conditions of a subject, including fever status and comfortability, to ensure full consideration of a subject's well-being.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is related to U.S. application ______, filed on ______, having an attorney docket number MAS.1247A, titled “Non-Contact Core Body Temperature Measurement Systems and Methods” and U.S. application Ser. No. 16/546,667, filed on Aug. 1, 2019, titled “Core Body Temperature Measurement,” entireties of which are incorporated by reference herein.

### TECHNICAL FIELD

The systems and methods disclosed herein are directed to obtaining and monitoring core body temperature, and, more particularly, utilizing such information to assist subjects in receiving quality care based on changes in their core body temperature or alternatively, stabilizations thereof.

### BACKGROUND

Core body temperature is the internal temperature of a body as opposed to the surface or skin temperature at the periphery. Significant core temperature changes over a short period of time can be detrimental to the body maintaining critical life-sustaining functions.

FIG. 1A shows a picture of a human body 100 that has varying degrees of heat generated and flowing throughout the core of its body. The temperature of an organism's body is maintained within a specific range in order for the body to carry out its essential functions. In some examples, the core temperature of the body may not uniform as shown in FIG. 1A. For example, the core body temperature increases towards the center of a person's body and decreases toward the outer extremities as shown in the arms and legs of body 100. The reason for this is because the pulmonary artery of the heart, shown in FIG. 1B, is what regulates the temperature of the body and thus, it is changing temperature of the pulmonary artery that influences other changes throughout the body. Thus, the temperature of the pulmonary artery has been accepted as the “gold standard” for measuring one's estimated core body temperature. In general, this also relates to the temperature of the central organs and the brain as shown in FIG. 1A.

“Clinical temperature” is the temperature that doctor's typically measure using a thermometer, which is an approximation of a subject's core temperature, but is not a true measurement of the core temperature itself. That is, “clinical temperature” is an adjusted proxy that is meant to match core temperature or approximate it as close as possible, but is not the actual temperature of the pulmonary artery of the heart for example. Clinical temperature is normally obtained by measuring the temperature of a subject's armpit, rectum or under a subject's tongue. In addition, not only will this temperature have a delta from core temperature, it will also have a different response time unique to the site of the body where the temperature measurement is obtained. This is because the pulmonary artery is always moving blood and is adept at adjusting temperature fairly quickly and thus, it takes time for the profusion to reach specific sites of the body to reflect that change in temperature.

Skin or surface temperature is the temperature of a subject's skin or outer surface. This temperature varies greatly from core temperature depending on the location of the subject, perfusion, other ambient conditions, as well as the physiology of the subject itself (including, for example, thickness of skin, etc.). Skin temperature may be obtained using a temperature measurement device from one of three classes: single point measurement, heat flux measurement, and zero heat flux measurement. These measurement classes can be further categorized as relating to active or passive devices and devices that provide steady-state measurements (equilibrium) or transient measurements.

Doctor's and other healthcare professionals have a great need for understanding changes to the body under different conditions. Temperature measurements can provide an indication as to whether the body is attempting to thermo-regulate, combat an infection, and so on.

### SUMMARY

The systems and methods for obtaining and monitoring estimated core body temperature disclosed herein have several features, no single one of which is solely responsible for their desirable attributes. Without limiting the scope as expressed by the claims that follow, certain features of the temperature system will now be discussed briefly. One skilled in the art will understand how the features of the disclosed technology provide several advantages over traditional systems and methods.

One aspect relates to a method for estimating core body temperature, the method comprising receiving, using a temperature measurement device, a sample of air from a subject's lungs, measuring temperature of the sample of air, estimating a core body temperature measurement based at least in part on the measured temperature, receiving supplemental physiological data from the subject, refining the core body temperature measurement based at least in part on the supplemental physiological data, and displaying a reading of the refined core body temperature measurement.

The temperature measurement device can include an intubation tube.

The temperature measurement device can include a hand-held device.

The method further can include receiving, from another temperature measurement device, a surface temperature measurement for the subject as measured over time, and wherein the reading can include a plot of the refined core body temperature and surface temperature over time.

The refined core body temperature and surface temperature can be displayed with respect to a single display icon.

The plot can include a heat map showing a duration of time that the subject was experiencing a particular refined core body temperature and surface temperature measurement relative to the duration of time the subject was experiencing a different refined core body temperature and surface temperature measurement.

The supplemental physiological data can include one or more of: pulse oximetry data, respiration rate, blood pressure, perfusion index, surface temperature, circadian rhythm, and heart rate.

The method can further include signaling an alarm based at least in part on one or more of: the estimated core body temperature and the refined core body temperature.

The method can further include adjusting a control parameter of a medical device based at least in part on one or more of: the estimated core body temperature and the refined core body temperature.

Another aspect relates to a method for determining the condition status of a subject, the method comprising estimating, using a temperature measurement device, a core body temperature measurement for a subject, receiving, from another temperature measurement device, a surface temperature measurement for the subject, determining perfusion information of the subject, and determining a trend for the subject's condition based at least in part on the core body temperature measurement, the surface temperature measurement, and the perfusion information of the subject.

The trend can indicate that a subject's fever is improving or has broken completely.

The method can further include identifying a fever status based at least in part on the determined trend.

The method can further include classifying the fever status of a fever of the subject based at least in part on the determined trend.

The method can further include displaying a fever status to a user via a user interface based at least in part on the determined trend.

The method can further include controlling application of a fever reducing device based at least in part on the determined trend.

The fever reducing device can include medication administered via an intravenous line.

The method can further include determining the trend is further based on a change in surface temperature, core body temperature and perfusion index.

The method can further include determining the trend comprises monitoring the change in surface temperature, core body temperature and perfusion index over a predetermined timeframe.

The predetermined timeframe can be less than thirty seconds.

The method can further include determining the trend is further based at least in part on one or more of: pulse oximetry data, respiration rate, blood pressure, circadian rhythm, and heart rate.

The method can further include displaying, via a user interface, a fever status classification along with a reading of the core body temperature.

Another aspect relates to a temperature acquisition system comprising: a temperature measurement device; and one or more computer hardware processors configured to execute program instructions to cause the temperature acquisition system to at least: receive a first surface temperature measurement at a first location of a subject, receive a second surface temperature measurement at a second location of a subject distinct from the first location, determine a first perfusion index measurement at the first location of a subject, receive a second a perfusion index measurement at the second location of a subject, and determine a condition of the subject based at least in part on one or more of: the surface temperature measurements and perfusion index measurements at the first and second locations.

The program instructions can further cause the temperature acquisition system to determine a change in surface temperature the first and second locations of the subject; and determine a time delay difference between the change in surface temperature at the first and second locations.

The first and second surface temperature measurements can be received concurrently.

The condition of the subject can indicate that the subject is experiencing one or more of: internal bleeding, compartment syndrome or an infection.

The condition of the subject can indicate that the subject is experiencing one or more of: internal bleeding, compartment syndrome or an infection with respect to one or both of the locations.

The condition of the subject can be displayed to a user via a user interface.

An alarm can be provided to a user based at least in part on the condition of the subject.

Yet another aspect relates to a system as illustrated in the drawings and/or described herein.

For purposes of summarizing the disclosure, certain aspects, advantages and novel features are discussed herein. It is to be understood that not necessarily all such aspects, advantages or features will be embodied in any particular embodiment of the disclosed technology and an artisan would recognize from the disclosure herein a myriad of combinations of such aspects, advantages or features.

### BRIEF DESCRIPTION OF THE DRAWINGS

The following drawings and the associated descriptions are provided to illustrate implementations of the present disclosure and do not limit the scope of the claims.

FIG. 1 depicts an example healthcare environment where temperature measurement devices according to the present disclosure can be implemented.

FIG. 2 illustrates a flow chart of an example method for obtaining core body temperature using air from a subject.

FIGS. 3A-3C illustrate example embodiments and methods for measuring temperature using certain medical devices, including intubation tubes.

FIGS. 4A-4D illustrate example embodiments and flows for sampling a subject's breaths for temperature measurements.

FIGS. 5A and 5B illustrate flow charts of example methods for tracking a subject's condition as informed by the subject's changing temperature.

FIG. 6 illustrates a flow chart of an example method for providing compensation mechanisms or a calibration for other medical device equipment responsible for obtaining temperature measurements for a subject.

FIGS. 7A-7C illustrate flow charts of example methods for providing feedback based on temperature measurements according to various embodiments of the present disclosure.

FIGS. 8A and 8B illustrate flow charts of example methods for performing stereo thermometry.

FIG. 9 illustrates a flow chart of an example method for analyzing data from a subject to determine a condition of the subject.

FIG. 10A-10B illustrate example user interfaces and/or indicators that may be implemented in conjunction with temperature measurement devices or methods according to various embodiments of the present disclosure.

FIG. 11A-11C illustrate additional example user interfaces that may be implemented in conjunction with temperature measurement devices or methods according to various embodiments of the present disclosure.

FIG. 12 is a block diagram of an example embodiment of a temperature monitor computing system with which certain methods, devices and systems discussed herein may be implemented.

While the foregoing “Brief Description of the Drawings” references generally various examples of the disclosure, an artisan will recognize from the disclosure herein that such examples are not mutually exclusive. Rather, the artisan would recognize a myriad of combinations of some or all of such examples.

### DETAILED DESCRIPTION

Generally described, the present disclosure relates to temperature devices for measuring, monitoring, and otherwise reacting to changes in estimated core body temperature. In the healthcare industry, there are many ailments that afflict subjects that are difficult to assess or diagnose at an early stage. For example, a subject may have an infection or may be suffering from compartment syndrome, where the pressure within the muscles can build to dangerous levels. Accordingly, the subject may be suffering from a fever in which the onset of the fever and the moment it begins to subside can be difficult to assess. Thus, changes in core temperature can provide an extraordinary amount of information to a person attempting to diagnose the subject suffering from such ailments. Alternatively, the subject may simply be uncomfortable due to an abnormal skin or core body temperature, but may be unable to speak or communicate this discomfort to a care provider.

Core body temperature is an important medical vital sign that in some contexts, must be exactly measured as close as possible in order to provide the best care possible for a subject, such as a person undergoing surgery or receiving hospital treatment. In such contexts, miniscule changes in the subject's core body temperature can indicate a potentially lethal situation for the subject. For example, fluctuations during a brain surgery operation can indicate that the subject is not coping with the procedure and may require the surgeon to reevaluate the surgical strategy in real-time in order to stabilize the subject.

Methods of measuring core body temperature have included static measurements as obtained from an ear, catheter or rectal measurements. However, monitoring changes in temperature in real-time and with a small margin of error has presented many problems for those assessing subjects in a healthcare setting (such as, for example, practitioners, surgeons, etc.). Moreover, responding to those changes in real-time has presented challenges to those in the healthcare setting who may need to respond quickly or who may not know that there is, for example, a threat to a subject's core body temperature that needs to be assessed. Further, a person may not know how to augment or enhance such body temperature data through use of other physiological indicators, such as changes in skin perfusion, blood oxygen content, ECG signals, circadian rhythm information, blood pressure, etc.

The aforementioned problems, among others, are addressed in some examples by the disclosed core body temperature measurement device and methods for accurately obtaining estimated core body temperature, as well as by methods for correlating estimated core body temperature measurements with conditions of the body. Through implementation of devices, systems and methods discussed herein, the estimated core body temperature of a person may be measured and augmented based on other physiological information received from other sensors and devices as will be described herein. Providing users with information regarding changes in physiology relative to one another may assist users in recognizing when certain actions needs to be performed. For example, a doctor may introduce medication for a fever as soon as one is detected and then withdraw the medication when the fever breaks or subsides based on the correlation between various physiological measurements.

In addition, this information gathered from a subject may be intuitively displayed to the subject or to a person assessing the subject's well-being, for example. As would be appreciated by one of skill in the art, the collection and dynamic display of estimated core body temperature, as disclosed herein, represents a significant technological advance over prior implementations. Specifically, the disclosed techniques can identify and display a filtered selection of information for a user, resulting in increased efficiency in understanding how a body is operating.

In addition, the resulting analysis of the collected information (including, for example, estimated core body temperature, surface temperature, perfusion index, circadian rhythm, oxygen saturation) is presented in an intuitive user interface for the user. It has been noted that design of computer user interfaces that are useable and easily learned by humans is a non-trivial problem for software developers. The various examples of interactive and dynamic user interfaces of the present disclosure are the result of significant research, development, improvement, iteration, and testing and in some examples, provide a particular manner of summarizing and presenting information in the realm of temperature measurement devices. This non-trivial development has resulted in the user interfaces described herein, which may provide significant cognitive and ergonomic efficiencies and advantages over previous systems. The interactive and dynamic user interfaces include improved human-computer interactions that may provide reduced mental workloads, improved decision-making, reduced work stress, and/or the like, for a user. For example, user interaction with the interactive user interfaces described herein may provide an optimized presentation of estimated core body temperature compared to surface temperature information and may enable a user to more quickly access, navigate, assess, and utilize such information than with previous systems which can be slow, complex and/or difficult to learn, particularly to novice users.

Various aspects of the disclosure will now be described with regard to certain examples, which are intended to illustrate but not limit the disclosure. Although the examples described herein will focus, for the purpose of illustration, specific calculations and algorithms, one of skill in the art will appreciate the examples are illustrate only, and are not intended to be limiting. For example, although described in the context of a connected vehicle, the disclosed data transfer optimization techniques can be implemented in other mobile networked computing environments.

### Overview of Example Network Connected Core Body Temperature System

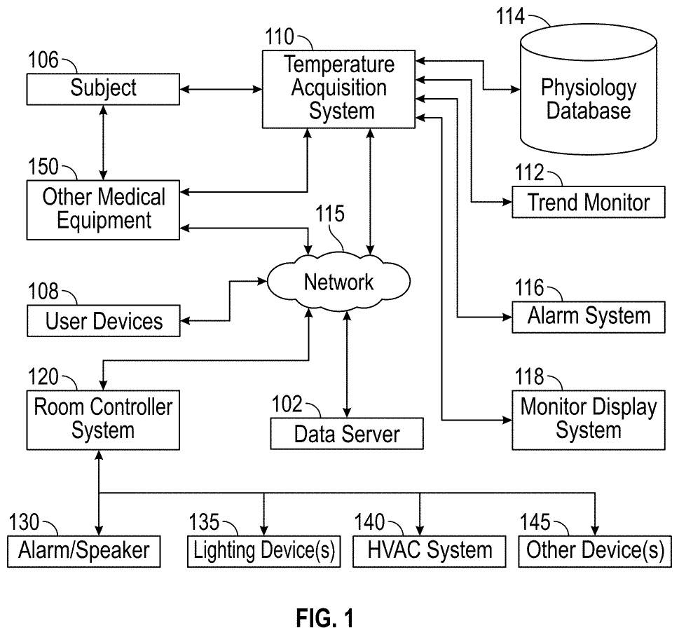

FIG. 1 depicts a schematic diagram of a network connected temperature acquisition system 110 in a healthcare environment in which various examples according to the present disclosure can be implemented. Healthcare environments can include any environment where health-related services, including hospital services, at-home hospice services, nursing home or other long-term care, etc., can be administered. FIG. 1 depicts a subject 106 that may be a patient of a hospital. For example, the subject 106 may be a person seeking care from a hospital, a pet at a zoo or in a veterinarian's office, etc. In a non-limiting example, the subject 106 may be an aquatic animal where the temperature acquisition system 110 may be fixed to the subject's blowhole to take a temperature reading of the animal's core body temperature. A user as described herein may refer to the subject 106 itself monitoring specific health related data or may refer to a healthcare professional. For example, the user as described herein may be a person with some relationship with subject 106 who may be caring for the subject, such as a doctor. In general, the user may be a person or entity using the physiological data of a subject 106 in order to care for a subject 106.

An end user device 108 may be any device capable of providing data to a user via a monitor display and/or speaker system. For example, end user device 108 may be a tablet, mobile device, laptop, desktop computer, virtual or augmented reality headset. In addition, end user device 108 may be connected to network 115 and capable of communicating with other devices over network 115. Alternatively, user device 108 may not communicate over network 115 and instead may receive data through an RFID device or a scanning device. In another example, user device 108 may have a touch enabled screen that is provided to subject 106 or a user 108 to allow the user of the device to control aspects of a medical device or room. The user device 108 may also receive data from medical device equipment 150 or temperature acquisition system 110. For example, in the example where end user device 108 is an augmented reality headset, a user may be able to view the estimated core body temperature of a person through the augmented reality headset in real-time as temperature acquisition system 110 monitors the data. In this way, a doctor may be able to see areas of a subject's body that have an abnormal estimated core body temperature and may receive audible queues through a speaker based on the temperature reading. For example, a user of user device 108 may receive an audible queue when a subjects 106 has broken based on an analysis of their physiological symptoms (including, for example, core temperature, skin temperature, perfusion, blood oxygen saturation, etc.) as described herein.

The devices, equipment and systems of FIG. 1 can establish different types of wireless connections, including satellite connections, WiFi connections, Bluetooth connections, RFID transmissions or data sharing, etc. The network 115 can include any appropriate network, including a private network, personal area network, an intranet, local area network, wide area network, cable network, satellite network, cellular network, peer-to-peer network, cloud network, etc. or a combination thereof, some or all of which may or may not have access to and/or from the Internet. The data network may be implemented by data servers, such as data server 102. Data server 102 and the other devices, equipment and systems of FIG. 1 may be remote from the temperature acquisition system 110. In some examples, data servers may be implemented on one or more host devices, such as blade servers, midrange computing devices, mainframe computers, desktop computers, or any other computing device that may provide computing services and resources.

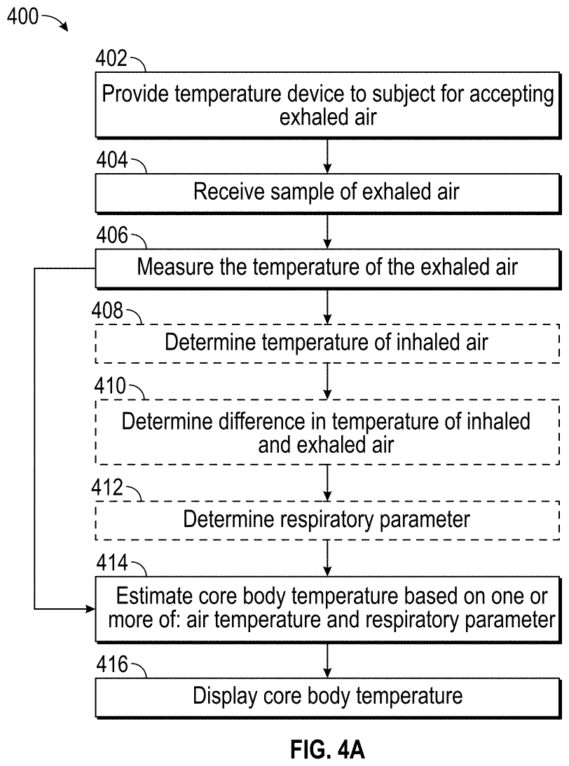

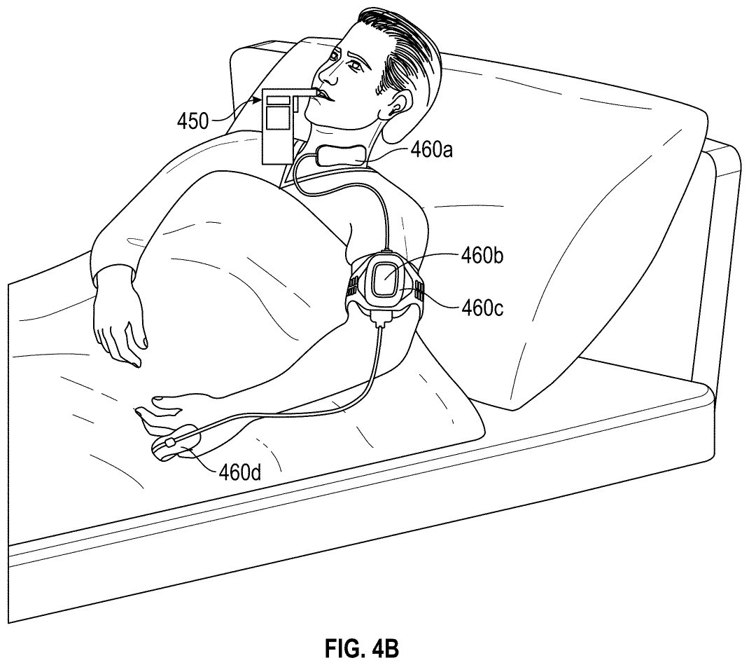

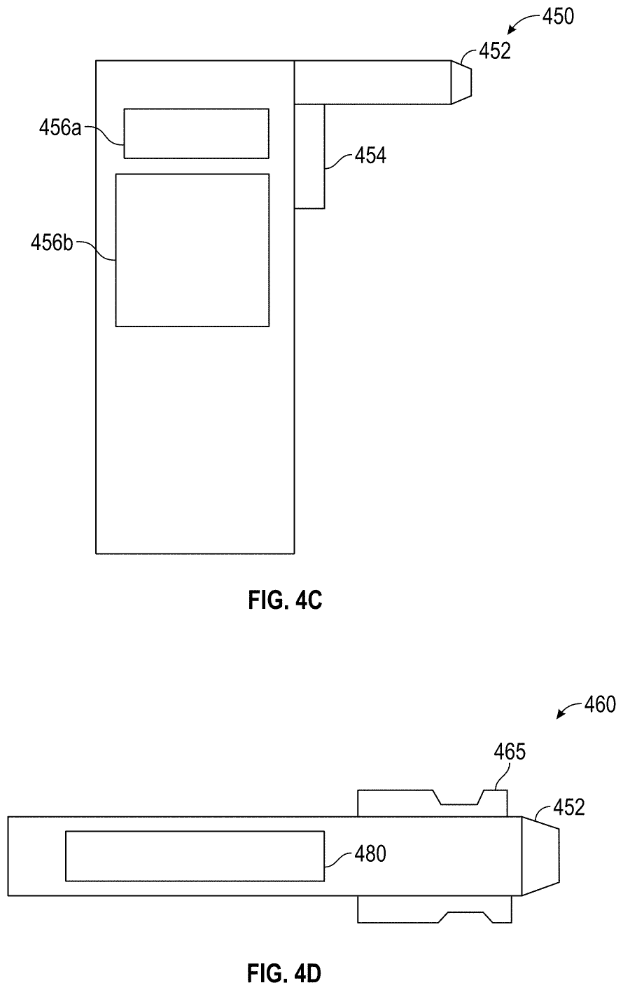

In the healthcare environment, the subject 106 may interface with a temperature acquisition system 110. For example, the subject 106 may be connected to the temperature acquisition system 110. Alternatively, temperature acquisition system 110 may be a contactless temperature measurement device that may take a temperature reading on a body without making contact with the body. In other examples, temperature acquisition system 110 may be a non-invasive device that makes contact with a subject to take a temperature measurement but, for example, does not require the subject to be connected to a machine or other monitoring device. For example, temperature acquisition system 110 may be a thermometer that takes a temperature reading from a subject's ear or across a subject's forehead. In another example, temperature acquisition system 110 may be a wearable device, such as a watch, bracelet, headband, earbud, etc. Temperature acquisition system 110 may be a handheld, portable device as in reference to examples of FIGS. 4B-4D. As described herein, a temperature acquisition system 110 may measure the estimated core body temperature. In some examples, temperature acquisition system 110 may concurrently measure the surface temperature of subject 106 or receive surface temperature information from another temperature measurement device that may measure surface temperature of subject 106. For example, temperature acquisition system 110 may be in wireless communication with another device in order to receive data from the other device. In some examples, the subject 106 may be connected to other medical device equipment 150, such as a pulse oximeter, electrocardiogram (ECG) or electroencephalogram (EEG) monitor, an intravenous (IV) line, contactless temperature reader, perfusion index calculator, infusion pump, etc. Similarly, the other medical device equipment 150 may transmit data to the temperature acquisition system 110 for further processing or over network 115 to another remote device. Alternatively, temperature acquisition system 110 may retrieve this data from a network data server 102 that processes and stores data received from other medical device equipment 150 over network 115.

As used herein, the term “remote” does not necessarily mean that two devices must be in different physical or geographic locations. Rather, remote devices include devices that communicate via a network connection, such as the Internet or a local area network (“LAN”), rather than devices that are directly connected via a wired or wireless connection. While devices in different physical or geographic locations may be remote devices, devices in the same location and devices physically operated by the same user may also be remote devices.

Temperature acquisition system 110 may include other systems, monitors, and databases, including physiology database 114, trend monitor 112, alarm system 116, monitor display system 118, etc. A person skilled in the art will recognize that these may not be physically part of the temperature acquisition system 110 and may be remote from temperature acquisition system 110. For example, physiology database 114 may not be physically connected to a temperature reading device 110, such as those described herein, and instead may receive data from the system wirelessly.

Physiology database 114 comprises any database or data structure (and/or combinations of multiple data structures) for storing and/or organizing data, including, but not limited to, relational databases (including, for example, Oracle databases, MySQL databases, and the like), non-relational databases, in-memory databases, spreadsheets, as comma separated values (CSV) files, extensible markup language (XML) files, TeXT (TXT) files, flat files, spreadsheet files, and/or any other format for data storage. For example, the content segments may be laid out as individual, structured) ML segments. Databases are typically stored in one or more data stores. Accordingly, each database referred to herein is to be understood as being stored in one or more data stores. In various examples, outgoing requests and/or incoming responses may be communicated in any suitable formats. For example, XML, JSON, and/or any other suitable formats may be used for API requests and responses or otherwise. As described herein, data transfer refers to both transmitting data from a connected vehicle over a network and receiving data at the connected vehicle over a network. The data may be in various formats, such as a database format, files, XML, HTML, RDF, JSON, a file format that is proprietary to the system, data object format, or any other format, and may be encrypted or have data of any available type.

Databases are typically stored in one or more data stores. Accordingly, each database referred to herein is to be understood as being stored in one or more data stores. In some examples, data server 102 and physiology database 114 may be implemented as a single entity. In a non-limiting example, data server 102 may comprise a single-node database server, such as an Oracle server.

Physiology database 114 may store data related to subject 106 including temperature data, oxygen saturation data, perfusion data, blood pressure, skin temperature, perfusion information, and the like. In addition, physiology database 114 may store trends and relationships between data items for a subject 106.

In addition, physiology database 114 may include data for multiple subjects 106. For example, physiology database 114 may be in communication with network 115 and receive data from other machines or devices taking measurements for other subjects. In this way, data server 102 or trend monitor 112 may extract patterns from physiology database 114 for the subject 106 or for multiple subjects such that the data may be more meaningful for the end user to review. In one example, a trained ML model can be used to process and analyze the physiology information in accordance with certain examples of this disclosure where ML models are considered advantageous (including, for example, predictive modeling, inference detection, contextual matching, and the like.). Examples of ML models that may be used with aspects of this disclosure include classifiers and non-classification ML models, artificial neural networks (“NNs”), linear regression models, logistic regression models, decision trees, support vector machines (“SVM”), Naive or a non-Naive Bayes network, k-nearest neighbors (“KNN”) models, k-means models, clustering models, random forest models, or any combination thereof. These models may be trained based on data indicating how users interact with query results. For example, certain aspects of the disclosure will be described using events, symptoms, or conditions (such as the onset of a fever) with respect to subjects and data received from the subject regarding their physiological condition including, for example, core temperature, skin temperature, perfusion, and the like.), for purposes of illustration only. In a non-limiting example, data server 102 or trend monitor 112 may receive data from one or more subjects 106 relating to core temperature and skin temperature as measured over time, which may be used to train an ML model for diagnosing the subject as suffering from an ailment, including a fever, discomfort, an infection, etc. or for diagnosing the improvement in an ailment, including the elimination of an infection or a fever. This helpful data may be shared across network 115 so that optimal results may be presented to more than one user based on their interaction with similarly situated subjects 106. For brevity, these aspects may not be described at length with respect to training an ML model as the particular mechanisms for training certain ML models are known in the art.

In some examples, a trend monitor 112 may interface with temperature acquisition system 110 in order to monitor trends in a subject's physiological data. For example, trend monitor 112 may deploy a ML model that is trained on a correlation between estimated core body temperature and a subject's circadian rhythm. Trend monitor 112 may receive this data from physiology database 114 or directly from another medical device 150 that is monitoring subject 106 in real-time. Trend monitor 112 may work in tandem with data server 102 to detect a threat to a subject's core body temperature. In a non-limiting example, trend monitor 112 may monitor a trend between surface temperature and core temperature during a surgery and may detect an abnormal change in core temperature that may require the surgeon's immediate attention.

Similarly, temperature acquisition system 110 may include an alarm system 116 that may provide an audible or visual alert that there is a change with the subject that may require attention or may indicate the swift recovery of a subject. For example, an alarm may be provided at the moment when temperature acquisition system 110 determines that a subject's estimated core body temperature is no longer destabilized in a certain area of the body or with respect to the entire body. The alarm system 116 also characterizes certain types of ailments, such as a type of fever or infection, based on temperature acquisition system's analysis of subject 106 data.

Temperature acquisition system 110 may also include a monitor display system 118. This may include any device that may display data including a touch enabled display. In some examples, the monitor display system 118 may be included with the temperature acquisition system 110. For example, a temperature measurement device may have an LED or LCD monitor on the device to display information directly on the device. In addition, the device may display information received over network 115. For example, temperature acquisition system 110 may take a temperature measurement for estimated core body temperature and send that information to data server 102 or trend monitor 112 wirelessly for further processing. The temperature measurements may be analyzed for patterns and indications of an abnormal condition or discomfort with the subject. Alternatively, the trend monitor 112 may be integrated with the temperature measurement device and may perform the processing on the device itself. In this example, the data server 102 may transmit data from physiology database 114 to the temperature acquisition system 110 such that trend calculations may be performed as part of the temperature measurement device. Monitor display system 118 may display static information or dynamically generated content. For example, monitor display system 118 may display a heat map or a graph showing the relevant vital signs. In a non-limiting example, monitor display system 118 may have multiple displays where one shows static information and another provides a graph in real-time showing changes in skin temperature and core temperature as temperature acquisition system 110 is obtaining those measurements.

The healthcare environment may include a room controller system 120 in wireless communication with other devices and systems of the healthcare environment. In some instances, room controller system 120 may include an alarm/speaker 130, lighting devices 135, heating, ventilation, and air conditioning (HVAC) systems 140 and other room controlling devices 145, such as Internet of Things systems. In some examples, alarm/speaker 130 may be provide any alarms or audible information to those in the room. For example, the alarm/speaker 130 may indicate low blood sugar for a subject or may indicate that it is time for a healthcare provider to visit the subject in the room. Lighting devices 135 may include the lighting mechanisms of an environment, including wirelessly controlled lightbulbs or other fixtures. In some instances, the status of a subject's ailment may correspond to a lightbulb setting so that a person may visualize their ailment as it subsides. For example, a subject's fever may be indicated by a red light that continues to grow dimmer as the fever subsides. In this way, a subject may visualize their fever as it disappears based on information obtained from temperature acquisition system 110 and other medical device equipment 150. In some instances, this light fixture may be a small LED bulb located on a device in the room or may be located separate from the subject's room. The lighting device 135 may also track a person's circadian rhythm or other condition of the subject, such as blood oxygen saturation levels or a heart rate monitor.

In addition, other devices 145 may include IV lines, infusion pumps, television sets, and the like controlled by the room controller system 120. Other devices 145 may also correspond to passive devices such as a room thermometer for controlling the HVAC system 140.

A user device 108 may control one or more of these room control devices. Alternatively, the room controller system may have automated controls such that the HVAC system is adjusted based on a reading from the subject. For example, if the subject is experiencing discomfort due to exposure to an uncomfortable temperature in the room, the HVAC system 140 may automatically respond to such an indication by adjusting the room temperature to one that would be better suited for the subject 106. A person skilled in the art would appreciate that the features described with respect to a room controller system 120 may be implemented separately from a room controller system 120. For example, the lighting device 135 may be a small LED indicator located on a bed in a hospital room but may not necessarily be controlled by a room controller system 120, where it may be unnecessary to control such a light over a network 115.

As described above, data server 102 may be a remote server that may perform one or all of the functions described herein including providing input to the room controller system or receiving data from user device 108, medical equipment 150, or temperature acquisition system 110. A person skilled in the art will appreciate that multiple data servers 102 may perform these functions in tandem for returning results in a robust and efficient manner.

In a healthcare environment as described above with respect to FIG. 1, different protocols and algorithms may be employed to monitor the well-being of subjects. As will be described below, various algorithms may be employed to provide the most helpful information for a user providing healthcare services to a patient, such as in a hospital operating room, delivery room, hospice care center, in a subject's home, and the like. The algorithms described below are designed to obtain accurate temperature measurements from a subject and determine when a patient is experiencing less than ideal conditions that may require additional attention. In addition, certain devices are described below that may obtain data from a subject for processing.

Measuring Core Body Temperature from Exhaled Air

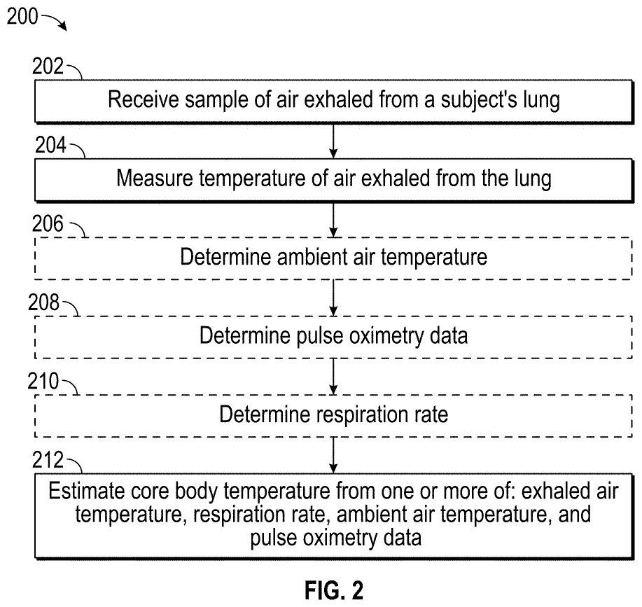

FIG. 2 depicts a flow chart illustrating an example logical flow of a method 200 for measuring estimated core body temperature based on characteristics of a subject's breath. In some examples, the method 200 may also incorporate other data points in order to refine or otherwise enhance the core body temperature estimate. The method 200 can be implemented at least partly within the healthcare environment of FIG. 1 and may be performed by temperature acquisition system 110.

At block 202, the temperature acquisition system 110 receives a sample of air exhaled from a subject's lungs. In this example, temperature acquisition system 110 may be a device that may accept air exhaled from the lungs of a subject 106. The device may include a compartment for receiving air or may allow air to flow proximate to a sensor as the air passes through the device.

At block 204, the temperature acquisition system 110 may determine characteristics of the air exhaled from the lungs, including moisture content, temperature, and the like. In some examples, sensors may be included with the temperature acquisition system 110 that can take temperature measurements of the subject's air or from within the subject's air passageway. In other examples the temperature sensor may be located on a device located within a cavity or orifice of the subject's body, such as with an internal pacemaker device or with an intubation tube as described herein. In yet another example, temperature acquisition system 110 may determine the exhaled air temperature from a subject providing a sample of air to an external device as described herein. In some examples, temperature acquisition system 110 uses one or more of a: thermometer, thermoelectric generator, resistance thermometer, thermistor, thermocouple, and the like, for measuring the temperature of a subject's exhaled air. Temperature acquisition system 110 may store this data, along with all other data collected by this method, in physiology database 114, for example, for use in trend monitoring or performing future estimations of core body temperature for the subject or another subject also part of the network.

At block 206, the temperature acquisition system 110 may optionally receive or determine information regarding the ambient temperature. For example, temperature acquisition system 110 may measure the ambient temperature near the area where the temperature measurement of block 204 is taken. For example, if the temperature sensor is placed on a subject's skin, the ambient temperature may refer to the ambient temperature at that location near the subject's skin. In other examples, the ambient temperature may simply refer to the temperature of the room in which the subject is located. Ambient temperature measurements assist the temperature measurement device in providing a refined and accurate reading of estimated core body temperature and changes in estimated core body temperature. In addition, temperature acquisition system 110 may receive the ambient temperature from another medical device, for example, over a network connection or through a direction connection.

At block 208, the temperature acquisition system 110 may optionally determine pulse oximetry data. In addition, temperature acquisition system 110 may receive the ambient temperature from another medical device, for example, over a network connection or through a direction connection. For example, a subject may have a pulse oximeter measuring the subject's blood oxygen levels and transmitting data to the network data server or transmitting the data directly to the temperature acquisition system 110. In such examples, the temperature acquisition system 110 can receive data transmissions that include SpO₂ measurements and ambient temperature measurements from other devices. This data can be used to estimate an accurate temperature reading of a subject's estimated core body temperature in conjunction with data received from other medical devices (including, for example, external skin temperature readings, perfusion information, respiratory rate, and the like.) or may be utilized on its own without aid from other information received that relates to estimated core body temperature. In other examples, the temperature acquisition system 110 may receive ambient air temperature information from a device other than a technical medical device, such as a room thermometer connected to the network.

At block 210, the temperature acquisition system 110 may determine the respiration rate of a subject. In some examples, determining the respiration rate of a subject is optional to determining the estimated core body temperature. In examples where respiration rate is used, temperature acquisition system 110 may be able to provide a more accurate estimation of core body temperature. Respiration rate may be measured by counting the number of breaths a subject takes in a period of time. For example, the temperature acquisition system 110 or another medical device may count the number of breaths per minute, every 30 seconds, every 2 minutes, and the like. In some examples, respiration rate may be determined from pulse oximetry data gathered in block 208. In some examples, an indicator may be associated with the subject's respiration rate based on characteristics of the subject (including, for example, age, gender, weight, and the like.). This indicator may be based on a threshold that is set for a number of different categories and may be displayed to the user (including, for example, normal, abnormal, labored, and the like.).

At block 212, the temperature acquisition system 110 may estimate the core body temperature of the subject based on the sample of air received. For example, the temperature acquisition system 110 may estimate the core body temperature based on the temperature of the subject's exhaled air. In another example, the temperature acquisition system 110 may estimate the core body temperature based at least in part on one or more of the exhaled air temperature, the respiration rate, the ambient air temperature, and pulse oximetry data. In yet another example, temperature acquisition system 110 may estimate core body temperature from characteristics of the subject's air and from there, may refine the estimate based on one or more of the subject's other physiological measurements. In a non-limiting example, temperature acquisition system 110 may estimate core body temperature from exhaled air temperature and then apply a bias to the estimate based on ambient air temperature as a corrective factor for a more robust core temperature measurement.

In various implementations, the described blocks of the flowchart of FIG. 2, and the other flowcharts described herein, may be performed in an order other than that specifically disclosed, or multiple blocks may be combined in a single block. The example blocks may be performed in serial, in parallel, or in some other manner. Further, blocks may be added to or removed from the disclosed example examples.

Measuring Estimated Core Body Temperature from Exhaled Air Using an Intubation Tube

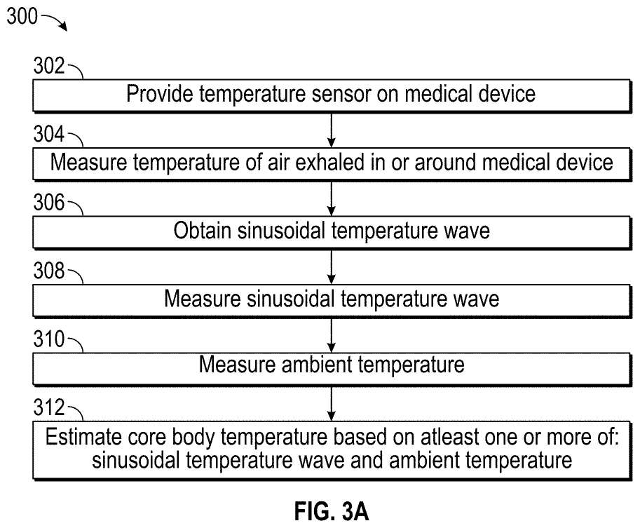

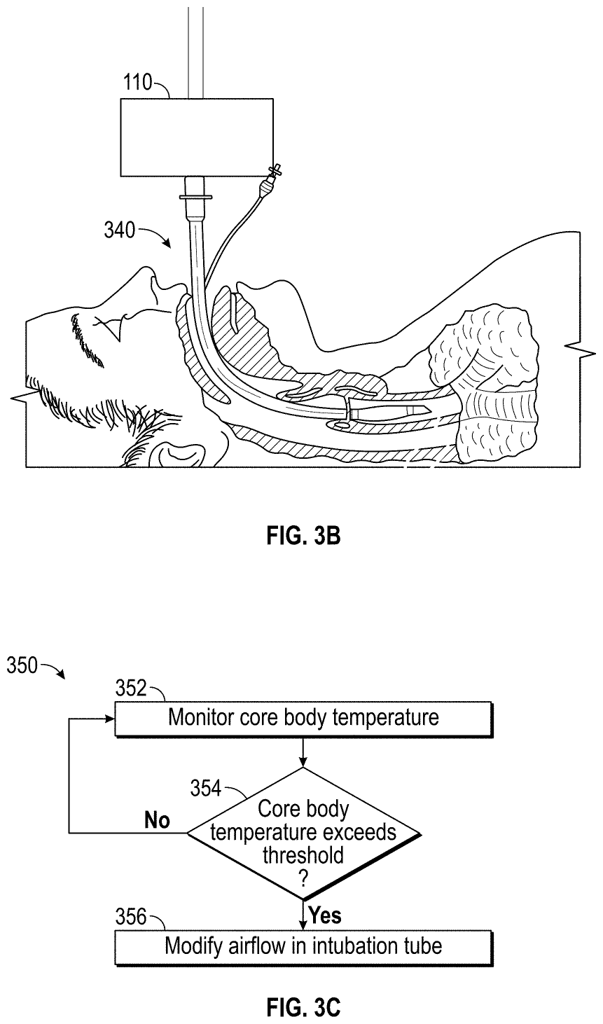

FIG. 3A depicts a flow chart illustrating an example logical flow of a method 300 for measuring estimated core body temperature using a temperature sensor coupled with an intubation tube. The method 300 can be implemented at least partly within the healthcare environment of FIG. 1 and may be performed by temperature acquisition system 110.

At block 302, a temperature sensor may be provided with a medical device. The temperature sensor may be a thermometer, thermoelectric generator, resistance thermometer, thermistor, thermocouple, and the like. The temperature sensor may be integrated with the medical device. In a non-limiting example, the temperature sensor may be integrated with an intubation tube that is inserted into a subject's air passageway. The temperature sensor may be located within the air passageway. In addition, the temperature sensor may be located within the medical device. For example, the temperature sensor may be located within a pacemaker or intubation tube that are in wireless communication with a remote device or data server.

At block 304, the temperature sensor of temperature acquisition system 110 measures the temperature of the air exhaled in or around the medical device. In some examples, the temperature sensor may measure the temperature continuously or periodically. In some examples, the frequency may be more or less than one second. For example, temperature measurements may be received once every thirty seconds or once every half second.

At bock 306, the temperature sensor of temperature acquisition system 110 obtains a temperature waveform from the measured air temperature. In some examples, the temperature waveform may or may not take on the characteristics of being a sinusoidal waveform.

At block 308, the temperature sensor of temperature acquisition system 110 may then measure and analyze the temperature waveform. For example, the waveform may have peaks and troughs that relate to the breathing of the subject 106.

At block 310, the temperature sensor of temperature acquisition system 110 or another temperature sensor may optionally measure the ambient temperature surrounding the device. In addition, the temperature acquisition system 110 may determine the respiratory rate of the subject 106.

At block 312, temperature acquisition system 110 may then estimate the core body temperature of the subject using the temperature of the exhaled air. In addition, the estimated core body temperature measurement may be enhanced through use of the respiratory rate of a subject, the blood oxygen saturation, and the like.

An example of the type of device described above is an intubation tube 340 that is inserted in a subject's airway as in FIG. 3B. In such examples, a temperature sensor may be located at the subject's mouth opening or closer to one or both ends of the intubation tube, such as nearer to the lungs or outside of the mouth. In some examples, the intubation tube 340 may be coupled to the temperature acquisition system 110 such that air from the patient may flow through the intubation tube 340 and the temperature measurement system 110 and into the air.

FIG. 3C depicts a flow chart illustrating an example logical flow of a method 350 for modifying the airflow in an intubation tube based on a subject's estimated core body temperature. Integrating a temperature acquisition system 110 with an intubation tube provides one method of monitoring estimated core body temperature using the method 300 of FIG. 3A.

At block 352, temperature acquisition system 110 may monitor a subject's estimated core body temperature as described above with respect to method 300 of FIG. 3A. At decision block 354, temperature acquisition system 110 may determine that the estimated core body temperature has exceeded a predetermined threshold, either in the negative or positive direction. Thus, at block 356, if the estimated core body temperature does exceed the predetermined threshold, then the air flow in the intubation tube may be modified to more airflow or less airflow in order to stabilize the patient based on fluctuations in their estimated core body temperature.

Portable Device for Measuring Estimated Core Body Temperature from Exhaled Air

FIG. 4A depicts a flow chart illustrating an example logical flow of a method 400 for determining estimated core body temperature in connection with a temperature measurement device as shown in FIGS. 4B-4D.

At block 402, a subject may be provided a temperature measurement device for accepting exhaled air. The temperature measurement device may be a temperature measurement device 450 illustrated in FIG. 4B. In one example, the device may be removably attached to the subject's airway. For example, the device may be connected to a subject's nostrils to measure air from the nostrils. In another example, the temperature measurement device may be a handheld, portable device that a subject may blow forced air into. Alternatively, temperature measurement device may receive exhaled air passively while the subject breathes naturally. Optionally, temperature measurement device may be connected to a semi-stationary medical apparatus, such as a tabletop machine having a tube that the subject may periodically breathe into, a storage trolley, an IV stand pole on wheels, and the like. For example, the patient may have an air tube that is connected to a non-portable machine that is stationed by the subject's side, such that the subject may periodically breathe into the tube at set intervals. The subject may receive an alert that it is time to provide an air sample to the device. Otherwise, the device may require the subject to provide a constant supply of air samples, where only a portion of those air samples are used to estimate core body temperature, other ailments or to classify a condition of the patient.

In some examples, temperature acquisition system 110 may comprise temperature measurement device (or a suitable alternative to these devices for receiving air samples), where temperature acquisition system 110 may measure estimated core body temperature using a sample of air collected every 1 minute, 2 minutes, 5 minutes, and the like. In some examples, the user of the system may customize ranges or intervals for collecting this information based on specifics for each individual subject. Temperature acquisition system 110 may keep a running average, or other statistic for monitoring trends, using data obtained from the subject based on exhaled air characteristics, temperature of the exhaled air, respiration rate, pulse oximetry data, blood pressure, and the like.

At block 404, the temperature measurement device may receive a sample of the subject's exhaled air. At block 406, the temperature measurement device may measure the temperature of the exhaled air. For example, the temperature measurement device may use one or more of a: thermometer, thermoelectric generator, resistance thermometer, thermistor, thermocouple, and the like for measuring the temperature of a subject's exhaled air.

Optionally, at block 408, temperature acquisition system 110 may determine the temperature of the subject's inhaled air. Optionally, at block 410, temperature acquisition system 110 may determine a difference in temperature between the inhaled and exhaled air temperatures. Optionally, at block 412, temperature acquisition system 110 may determine the respiratory parameter of the subject. In some example, the respiratory parameter is respiratory rate.

At block 414, temperature acquisition system 110 may estimate the subject's core body temperature based at least in part on air temperature, respiration rate, and the like. In some examples, temperature acquisition system 110 may apply a transformation rule to the exhaled air temperature to estimate the core body temperature of the subject. Additionally, temperature acquisition system 110 may combine the data previously received, including respiration rate and inhaled air temperature to refine the estimated core body temperature measurement. In some examples, temperature acquisition system 110 may further refine the estimated core body temperature measurement using pulse oximetry data, perfusion data, surface temperature of the subject measured at one or more locations, circadian rhythm, blood pressure, blood oxygen saturation, inhaled air temperature, and the like. This data may be retrieved from physiology database 114 or directly from the other medical device equipment 150 measuring vital signs of the subject.

At block 416, the estimated core body temperature measurement may be displayed to the subject or to a user. For example, temperature acquisition system 110 may display the temperature as a static measurement, as a moving graph over time, or as a heat map as described herein.

FIG. 4B illustrates a sample temperature measurement device 450. Temperature measurement device 450 may be a device that a subject may blow into. In some examples, the sample temperature measurement device 450 may be a portable device. Temperature measurement device 450 may include one or more of a: thermometer, thermoelectric generator, resistance thermometer, thermistor, thermocouple, and the like for measuring the temperature of a subject's exhaled air.

In some examples, the temperature measurement device 450 may be used in conjunction with a patient monitoring device 460c. The patient monitoring device may, for example, monitor respiration of a patient. The patient monitoring device 460c may be Radius-7® available as Masimo Corporation, Irvine, Calif. The respiration monitoring device 460c may include a sensor 460a, a sensor 460d, and a display 460b. The sensor 460a may be placed about a patient's neck. The sensor 460a may be able to detect acoustic signals from the patient. The sensor 460d may be placed or applied, for example, on a finger of a patient. The sensor 460a may be able to measure various parameters including, but not limited to, total hemoglobin, carboxyhemoglobin, methemoglobin, oxygen content, pulse rate, oxygen saturation, perfusion index, and the like. Information collected by the respiration monitoring device 460c may be used in conjunction with the temperature measurement device 450 to better estimate the core body temperature.

As shown in FIG. 4C, device 450 may include a mouthpiece 452, a trigger 454, and displays 456a and 456b. The trigger may be optional where the device activates automatically upon detecting that forced air is entering the device. Otherwise, a user may be able to press the trigger before blowing into the device 450 for an accurate reading of estimated core body temperature. In some examples, the trigger may be a capacitive sensor or a touch sensitive sensor. In a non-limiting example, the trigger may be placed on the mouthpiece for activation by placement of the subject's mouth on the device. The display 456a may display the static number relating to the estimated core body temperature, whereas 456b may include more detail regarding the temperature reading including trends over time.

FIG. 4D provides another sample example for a device 460 that a subject can breathe into. The device 460 can include a mouthpiece 452, a lip guide 465, and a display 480. The device may accept airflow through the device. For example, the airflow may enter mouthpiece 452 and exit through the opposite end. In addition, lip temperature measurements may be received through lip guides 465. For example, the lip guide 465 may include a temperature sensor for measuring the ambient temperature as well as the subject's lip temperature once the lips are placed on the lip guide. In addition, lip guides 465 may measure other physiological data, including perfusion and blood oxygen saturation. The lip temperature corresponds to skin temperature which is utilized in examples described herein to provide information regarding how a subject's estimated core body temperature is fluctuating as compared to the surface temperature of the subject. For example, the surface temperature, core temperature, and perfusion information may be used to determine when a user's fever has broken or is subsiding.

### Determining and Tracking Condition Status

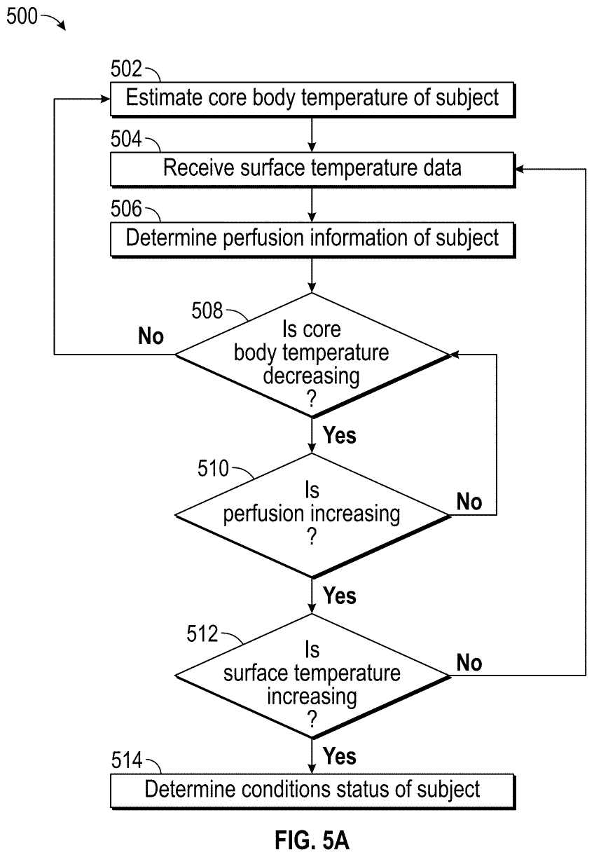

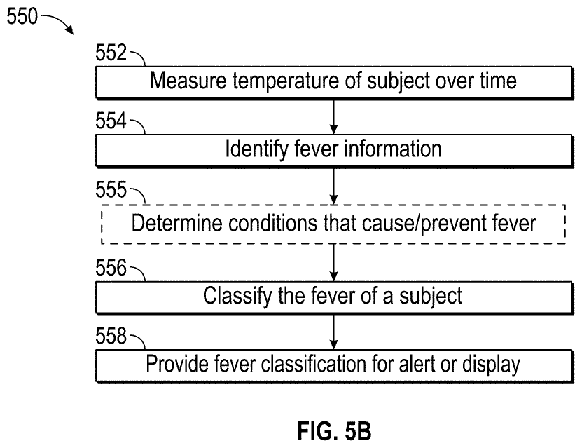

For example, FIG. 5A depicts a flow chart illustrating an example logical flow of a method 500 for determining the condition of a subject based on their physiological information. At block 502, a temperature measurement device of temperature acquisition system 110 estimates the core body temperature of a subject as described herein. At block 504, temperature acquisition system 110 receives temperature of the surface temperature. In one example, surface temperature may be measured through employing a four thermistor model.

At block 506, temperature acquisition system 110 determines perfusion information for the subject. In a non-limiting example, temperature acquisition system 110 will use information obtained from a pulse oximeter to determine a subject's perfusion index. At decision block 508, temperature acquisition system 110 may track changes in estimated core body temperature over time. For example, the temperature acquisition system 110 can measure the core and perfusion index of a subject once a minute, every 30 seconds, every 5 seconds, once in a 5 minute period, and the like. If the subject's estimated core body temperature is decreasing, then temperature acquisition system 110 may determine, at decision block 510, whether the perfusion index is increasing. If the estimated core body temperature is not decreasing (for example, changing or is increasing), then the method may revert to block 502 where the method continues to monitor and estimate the core body temperature of the subject.

If the perfusion index is increasing, then temperature acquisition system 110 may determine, at decision block 512, whether the surface temperature is increasing. If the perfusion index is not changing or is decreasing, then the method may revert to block 504 where the method continues to monitor changes in estimated core body temperature of the subject, for example, by receiving surface temperature data.

If the surface temperature is increasing, then temperature acquisition system 110, at decision block 512, may determine the condition status of the subject at block 514. For example, the condition status may be that a person's fever has broken.

FIG. 5B depicts a flow chart illustrating an example logical flow of a method 550 for tracking and classifying a subject's fever status. For example, a person may have a high, sustained fever or they may have an oscillating fever. At block 552, the temperature acquisition system 110 can measure the core and surface temperature of a subject over time. For example, the temperature acquisition system 110 can measure the core and surface temperature of a subject once a minute, every 30 seconds, every 5 seconds, once in a 5 minute period, and the like. Temperature acquisition system 110 may also receive data relating to perfusion, blood oxygen saturation, respiration rate, and the like. Based on one or more of the core temperature, surface temperature, perfusion index, blood oxygen saturation, respiration rate, and the like, temperature acquisition system 110 can identify, at block 554, fever information of the subject for classification. In some examples, a ML model may be trained on any or all of the above inputs to determine, among other things, the classification of a fever. For example, the fever may be classified as a high, sustained fever, oscillating fever, intermittent fever, and the like. An ML model may provide pattern recognition capabilities that can accurately classify a fever through an ML training process. The ML model may also be trained to determine when the fever has broken, estimated core body temperature, circadian rhythm, and the like. In another example, a lookup table or data library may be used with data relating to fever classifications, prediction models, and/or trends that allow temperature acquisition system 110, with or without an ML model, to classify the health status of a subject based at least in part on data obtained by temperature acquisition system 110.

Optionally, at block 555, the temperature acquisition system 110 may identify or determine various conditions that may cause or prevent fever. Such conditions may be helpful to understand as to why a subject is suffering from a fever.

Where a subject is exhibiting signs of a fever, temperature acquisition system 110, at block 556, can classify the fever of the subject. At block 558, temperature acquisition system 110 can then provide the fever classification to a display system, alert system or may store the classification data in a database for future reference.

### Calibration and Compensation System

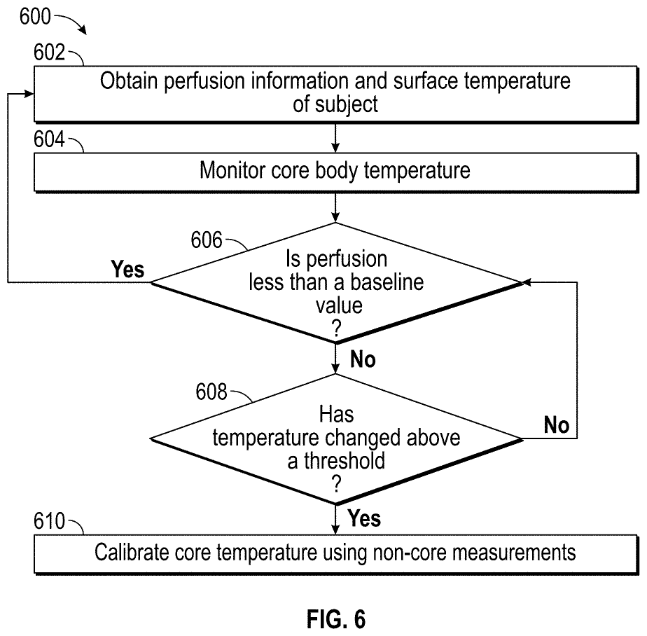

FIG. 6 depicts a flow chart illustrating an example logical flow of a method 600 for performing a calibration or compensation for medical devices attempting to estimate core body temperature. At block 602, a measurement device may obtain perfusion information from a subject. In addition, temperature acquisition system 110 may obtain the surface temperature of the subject. At block 604, a temperature measurement device can estimate and monitor the core body temperature of a subject. At decision block 606, temperature acquisition system 110 may determine whether the perfusion index is less than a baseline value. If the perfusion index is less than the baseline value, the method can revert to block 602. If the perfusion index is not less than the baseline value, than the method proceeds to block 608. At decision block 608, temperature acquisition system 110 may determine whether the core temperature has changed more than a threshold amount. If the core temperature has changed more than the threshold amount, the temperature acquisition system 110 may calibrate the core temperature using the perfusion and surface temperature measurements (i.e., the non-core measurements). If the core temperature has not changed more than the threshold amount, the method may revert to block 606.

In some examples, temperature acquisition system 110 may undergo a similar calibration method to fine tune the data readings based at least in part on data received from other tools or based on manual tuning from a health care professional or artificial intelligence adjuster. This calibration may be done in a factory or on the fly as necessary. Accordingly, temperature acquisition system 110 may be placed in a setup process where these adjustments may be made. Otherwise, temperature acquisition system 110 may perform a self-calibration during use without need of a setup process. For example, temperature acquisition system 110 may conduct a self-calibration on the network at a set time of the day when it is least likely to be used. The temperature acquisition system 110 may then perform micro adjustments throughout the day following an earlier adjustment.

### Alarm and Alert System

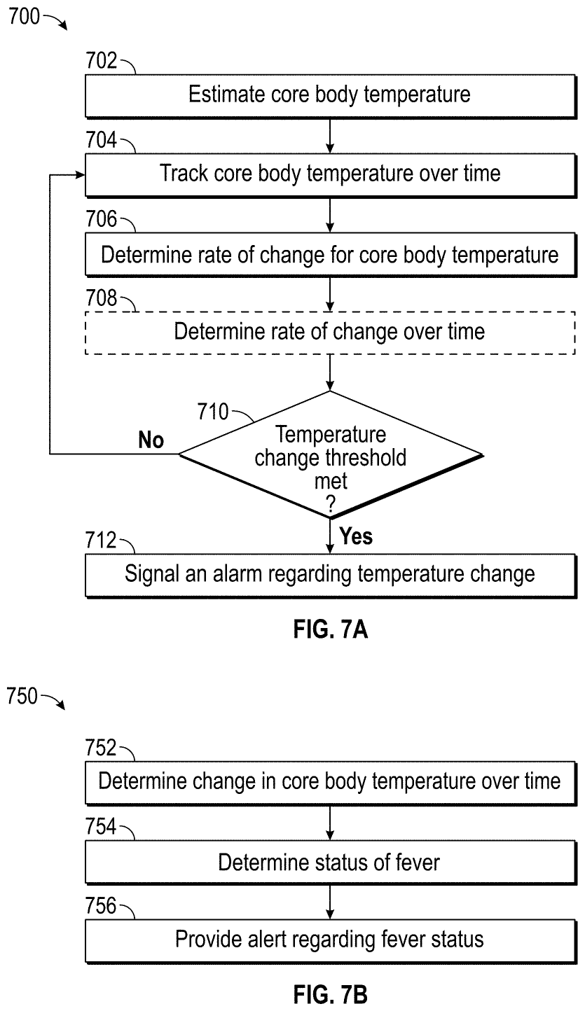

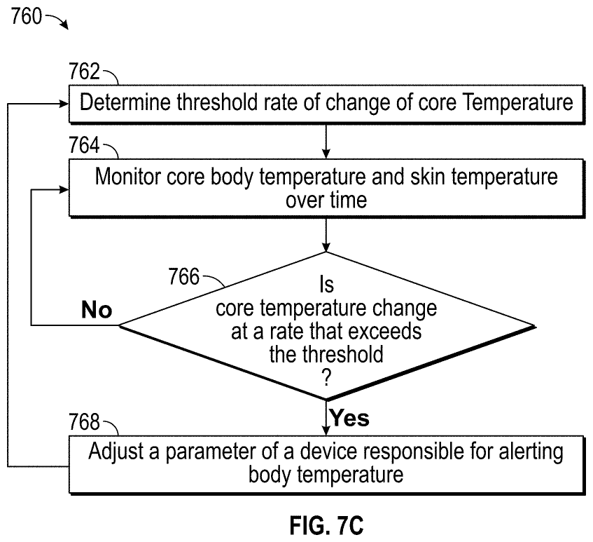

FIG. 7A depicts a flow chart illustrating an example logical flow of a method 700 for providing an alarm system that detected changes in estimated core body temperature. At block 702, temperature acquisition system 110 estimates the core body temperature of a subject. At block 704, temperature acquisition system 110 tracks the estimated core body temperature of a subject over time. At block 706, temperature acquisition system 110 determines a rate of change for estimated core body temperature. At optional block 708, temperature acquisition system 110 determines a rate of change for estimated core body temperature over time (including, for example, an acceleration or deceleration of the changing body temperature). At decision block 710, temperature acquisition system 110 determines whether the temperature change threshold exceeds a predetermined threshold. For example, the temperature change threshold may be predetermined as a change of half a degree in less than 30 seconds or could be an accelerating rate of change of 0.1 degrees per second squared in a 5 second period. Both thresholds may be used concurrently to complement one another for a more robust alarm system. For example, an alarm may be suppressed even though the rate of change threshold is satisfied where other conditions have not yet been satisfied, such as the rate of temperature change over time threshold or a perfusion index threshold. Alternatively, the alarm system may be triggered when either one of the rate of temperature change or the rate of temperature change over time exceeds a predetermined threshold. In other examples, the temperature change threshold may be dynamically determined using a ML model trained on data specific to each subject. The thresholds for these changes may vary based on a person's weight, gender, and the like. As such, the thresholds may adapt automatically so as to be unique for each individual subject. In a non-limiting example, the thresholds may be manually adjusted and automatically adjusted following a manual adjustment. The ML model may be trained to determine when a manual adjustment is necessary or when an automatic adjustment should suffice. If the temperature change threshold exceeds a predetermined threshold (for example, a problematic change is detected), temperature acquisition system 110 may signal an alarm regarding the change at block 712. For example, alarm system 116 may be signaled to provide an alarm that the core temperature has changed too much and is changing greatly in a short amount of time. The alarm may start out as a warning and then may escalate to a more drastic alarm signal. On the other hand, if the temperature change threshold does not exceeds a predetermined threshold (for example, a problematic change is not detected), the temperature acquisition system 110 may continue to track estimated core body temperature at block 712.

Similarly, FIG. 7B depicts a flow chart illustrating an example logical flow of a method 750 for providing an alarm based on the fever information as discussed in connection with FIGS. 5A and 5B. At block 752, temperature acquisition system 110 may determine a chance in estimated core body temperature and track the change over time. Temperature acquisition system 110 may also track if the rate at which the temperature is changing is speeding up or slowing down over time. At block 754, temperature acquisition system 110 may determine the status of a subject's fever. Determining the status or classification of a subject's fever may be done as discussed above in connection to FIGS. 5A and 5B. At block 756, an alert or alarm may be triggered based on the person's fever status. In some examples, an LED light may change colors gradually based on the status of a subject's estimated core body temperature or fever status.

In another example, a measurement of estimated core body temperature may trigger an action from another device. FIG. 7C depicts a flow chart illustrating an example logical flow of a method 760 for adjusting the parameter of another device based on estimated core body temperature measurements. At block 762, temperature acquisition system 110 may determine a threshold for the rate of change for estimated core body temperature. This threshold may be based on the output of an ML model trained on estimated core body temperature in various scenarios. In a non-limiting example, a subject may be receiving treatment through an infusion pump. Thus, changes in estimated core body temperature may signal that changes need to occur within the infusion pump to keep the subject from entering a critical condition. At block 764, temperature acquisition system 110 monitors the estimated core body temperature over time. In addition, temperature acquisition system 110 may optionally monitor other characteristics of the body, including surface temperature over time. Other measurements may also be obtained for perfusion, blood oxygen saturation, blood pressure, and the like. In some examples, these other measurements may be used to refine the measurement for estimated core body temperature. At block 766, the estimated core body temperature may be monitored to ensure the temperature does not change more than the threshold amount. At block 766, if core temperature does change at a rate that exceeds the threshold amount, then temperature acquisition system 110 may send a signal to adjust a parameter of a device responsible for altering the estimated core body temperature at block 768. If core temperature does not change at a rate that exceed the threshold, the temperature acquisition system 110 may revert to monitoring the code body temperature and skin temperature over time at block 764. For example, in the example of an infusion pump, feedback may be provided to the infusion pump to decrease flow based on an excessive change in estimated core body temperature.

### Stereo Thermometry

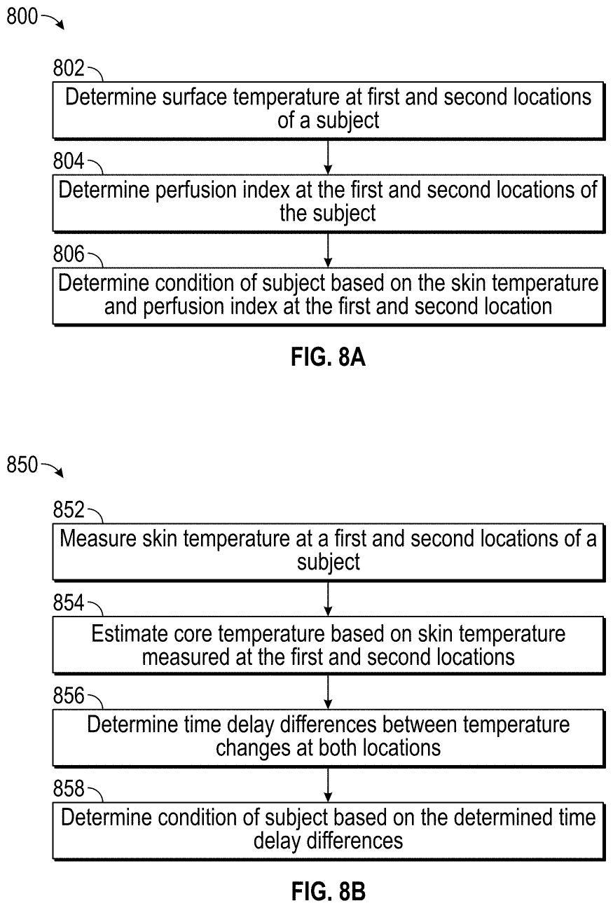

In another example, temperature acquisition system 110 may employ stereo thermometry to determine the condition of a subject. FIG. 8A depicts a flow chart illustrating an example logical flow of a method 800 for performing stereo thermometry on a subj ect.

At block 802, temperature acquisition system 110 may determine the surface temperature at different locations of the subject. For example, the different locations may be different appendages of the subject or different locations of the same appendage. In a non-limiting example, one location may be a predetermined distance from the pulmonary artery, such as on the chest of a patient, while the other one or more separate locations may be further away from the pulmonary artery, such as on the outer extremities of the patient's feet and hands. In some examples, the first location may be in reference to the location of a sensor on or within an intubation tube. In another example, the first location the first location may be in reference to the location of a sensor on or within a portable device.

At block 804, temperature acquisition system 110 may determine the perfusion index at those different locations of the subject. At block 806, temperature acquisition system 110 may then determine the condition of subject based on the skin temperature and perfusion index at the first and second locations. For example, temperature acquisition system 110 may determine that the subject is suffering from an infection or suffering from compartment syndrome based on the stereo thermometry measurements.

In another examples, FIG. 8B depicts a flow chart illustrating an example logical flow of a method 850 for performing stereo thermometry on a subject with time delay measurements. At block 852, temperature acquisition system 110 may determine skin temperature at a first and second locations of a subject. At block 854, temperature acquisition system 110 may estimate core temperature at the first and second locations of the subject. At block 856, temperature acquisition system 110 may determine the time delay differences between temperature changes at both locations. For example, the time delay differences can be on the scale of 0.5 seconds, 0.25 seconds, 1 second, 2 seconds, and the like before both locations are measuring the same surface temperature.

At block 858, temperature acquisition system 110 may determine a condition of the subject based on the determined time delay differences. In a non-limiting example, the time delay differences may be included in a lookup table or may be analyzed using a pattern recognition algorithm. Temperature acquisition system 110 may infer blood flow based on this information. In some examples, the blood flow inference may be used to diagnose certain ailments relating to blood flow. For example, temperature acquisition system 110 may determine from inferred blood flow that a subject may be suffering from compartment syndrome, where there may be internal bleeding following a surgery or tissue swelling in the subject's body. Monitoring estimated core body temperature changes using stereo thermometry may provide a health care professional with a faster indication that the patient is suffering from an ailment and based on the time delay differences may allow the health care professional to pinpoint exactly where the ailment is originating.

### Comfort Level Indicators

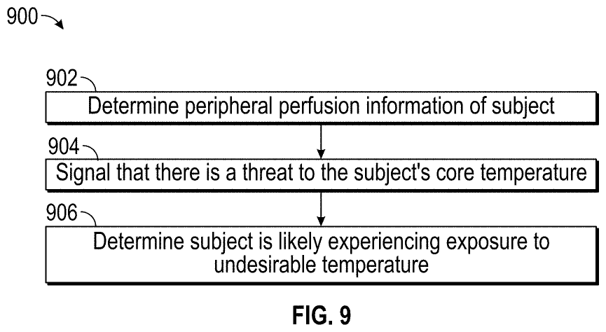

FIG. 9 depicts a flow chart illustrating an example logical flow of a method 900 for determining whether a subject is experiencing discomfort. At block 902, temperature acquisition system 110 may determine peripheral profusion information of a subject. At block 904, temperature acquisition system 110 may signal that there is a threat to the subject's core temperature based on one or more of the peripheral profusion information surface temperature information, estimated core body temperature information, and the like. At block 906, temperature acquisition system 110 may determine that the subject is likely experiencing exposure to an undesirable temperature. For example, the subject may be under a heavy blanket and may be too hot but may be unable to communicate adequately that they are hot. In some examples, this determination may be based on a comparison of the relative changes in estimated core body temperature, profusion and surface temperature. An alarm may be triggered that communicates to a caregiver that, for example, the heavy blanket should be removed from the subject due to the subject's physiological measurements. Temperature acquisition system 110 may continue to monitor the subject's physiological measurements to ensure that whatever change has occurred with the subject is resulting in a better comfortability reading from the temperature measurement device. A threat to core body temperature may also result in an automatic adjustment, such as an automatic adjustment of the HVAC system in the room, or may simply provide a call to another device signaling the discomfort reading.

### User Interfaces and Displays

The various examples discussed above result in a wide array of information relevant to a subject's condition (including, for example, core body temperature, surface temperature, perfusion index, and the like.). This data can then be provided to a user of this information, such as a doctor, surgeon, caregiver, or at-home nurse, such that they can take some action for the subject's well-being. For example, a doctor may need to provide a recommendation to the subject or alter a surgical procedure in-real time. Thus, a user interface that displays this information is beneficial to the user as they contemplate an action plan. On the other hand, presenting too much information to the user may overwhelm the user, where only a portion of the information is relevant. In order to provide a user with the most relevant information possible, the user interfaces may need to present a reduced amount of information so that the user can quickly study the data and take some action based on the data. In this way, a balance can be struck between providing too much information and only providing the most relevant data in a user friendly manner. Example user interfaces, charts, and graphs are described below that seek to strike this balance.

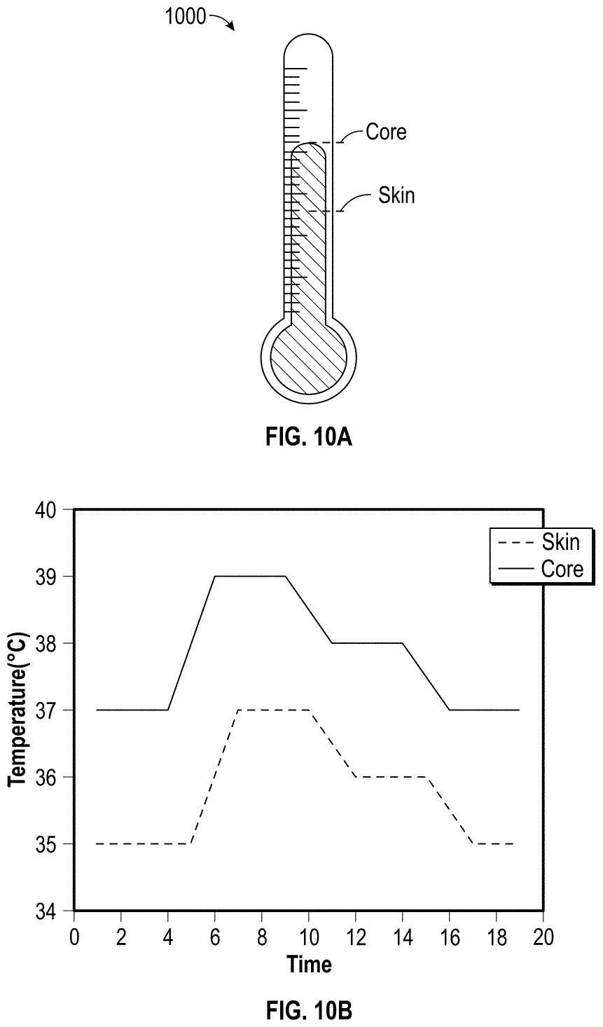

FIG. 10A depicts an example icon for an interactive user interface 1000 showing core temperature and skin temperature using the same icon. In a non-limiting example, the icon can be a thermometer as shown in the figure. Other appropriate icons may be used to display a juxtaposition of core temperature against skin temperature.

FIG. 10B depicts an example graph showing core temperature and skin temperature over time. This may be a dynamically changing graph that moves along the time axis as data is collected in real-time. Core temperature may be estimated as described herein. Skin temperature may be measured using a thermistor model, contact thermometer, non-contact thermometer, etc. Optionally, perfusion information described herein may be displayed to indicate the thermoregulation state in the body. For example, the perfusion information may be received from a pulse oximeter sensor or another device, such as a memory device. Changes in perfusion over time can be used with the changes in body temperature over time to calculate the estimated core body temperature.

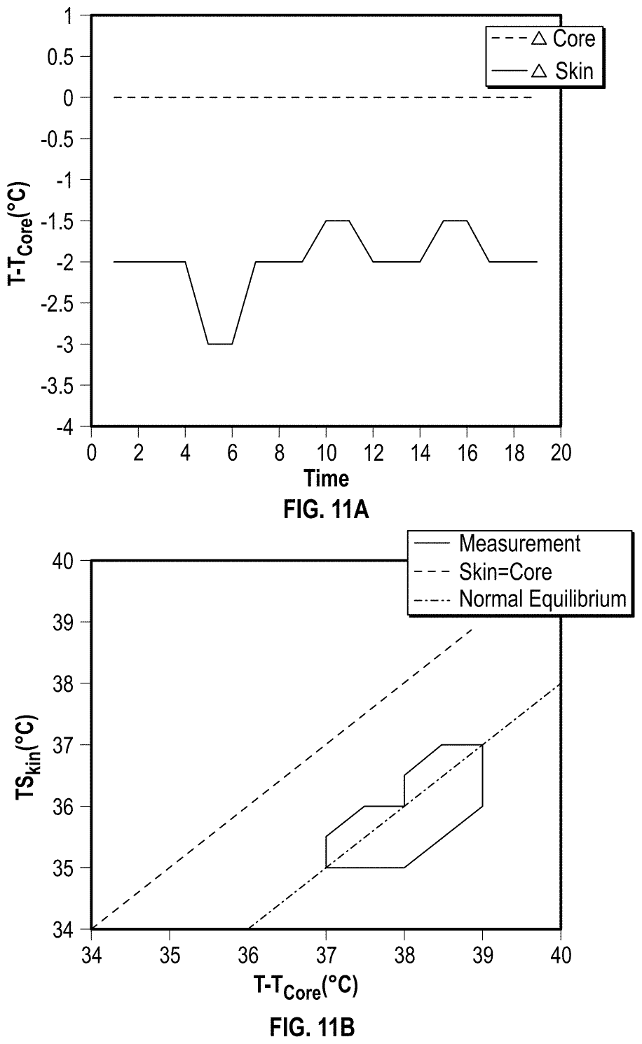

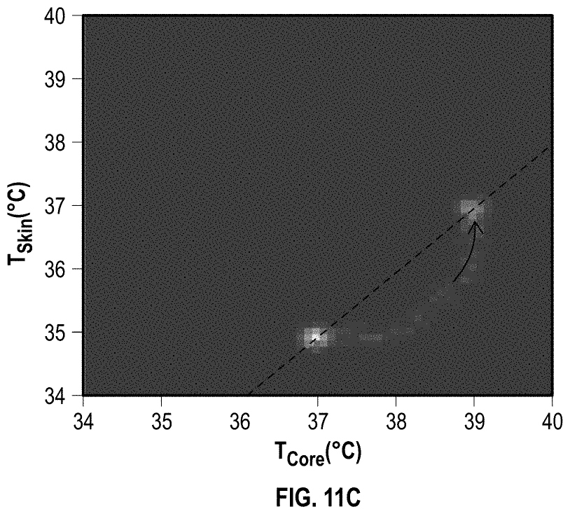

FIG. 11A depicts an example graph showing the different in skin temperature and core temperature over time.

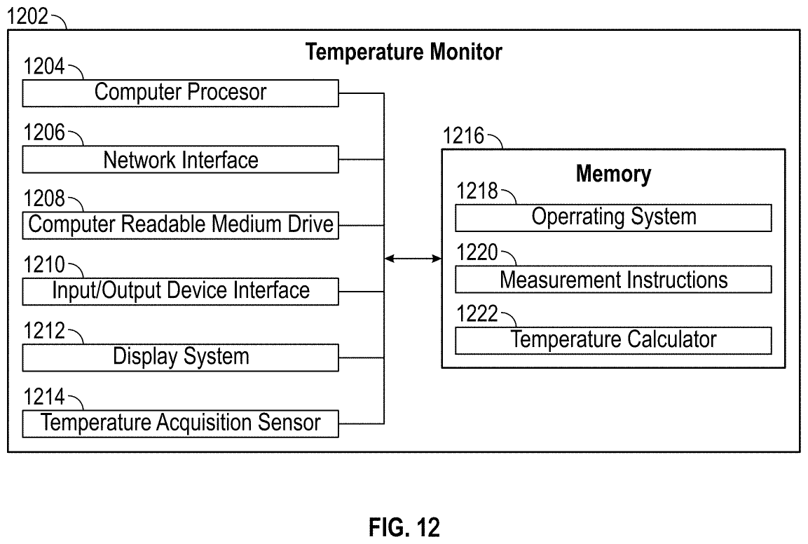

FIG. 11B depicts a similar graph to that of FIG. 12A except it plots the change over time with respect to a normal equilibrium. Using this information, a heat map can be generated that illustrates how much time is spent at each point depicted in FIG. 12B. For example, the data point in the top right where skin temperature is 37° C. and 39° C. may have been brief, whereas the time spent at the next data point may have been longer.

FIG. 11C depicts an example heat map corresponding to the graph of FIG. 11B. The heat map may illustrate how much time was spent at each stage of the loop shown in FIG. 12B. Optionally, the heat map may show directionality. For example, the arrow in the heat map indicates the directionality of the data points (for example, corresponding to the time flow).

In some examples, the temperature acquisition system 110 may display a graph tracking estimated core body temperature as it relates to circadian rhythm. Circadian rhythm may be used to achieve a better core body temperature estimate. In addition, changes in estimated core body temperature may be monitored over time to compare against the subject's normal circadian rhythm to detect abnormalities in the estimated core body temperature or in the patient's circadian rhythm over time.

### Execution Environment

FIG. 12 is a block diagram of an illustrative temperature monitor computing system 1202 to execute the processes and implement the features described above in connection with, for example, the systems, interfaces, and/or devices of FIGS. 1-13. In some examples, the temperature monitor computing system 1202 may include: one or more computer processors 1204, such as physical CPUs; one or more network interfaces 1206, such as a network interface cards (“NICs”); one or more computer readable medium drives 1208, such as high density disk drives (“HDDs”), solid state drives (“SDDs”), flash drives, and/or other persistent non-transitory computer-readable media; an input/output device interface 1210, such as an IO interface in communication with one or more external storage drives; one or more display systems 1212; one or more temperature acquisition sensors 1214, such as non-contact or contact temperature acquisitions sensor; and one or more computer readable memories 1216, such as RAM and/or other volatile non-transitory computer-readable media.

The one or more display system 1212 may comprise system software for generating user interface data based on the temperature measurements to be presented. The display system 1212 may comprise a LCD, LED, OLED, QLED, 4K UHD, plasma, and the like. The display device may be touch-enabled and receive touch input from a user. Display system 1212 may be further designate a display device within a healthcare environment or a mobile device associated with a user. Display system 1212 may receive input from the user devices notifying the display system 1212 of the size and shape of the display device and may format the user interface data accordingly. In some instances, a scroll feature may be provided when the display screen is not sized accordingly to fit all relevant content.

The computer processors 1204 can provide signal processing capabilities that condition and analyze temperature signals for output to the display system, alarm system, or other system that may convey information regarding a condition of a subject.

The network interface 1206 can provide connectivity to one or more networks or computing systems. The computer processor 1204 can receive information and instructions from other computing systems or services via the network interface 1206. The network interface 1206 can also store data directly to the computer-readable memory 1216. The computer processor 1204 can communicate to and from the computer-readable memory 1216, execute instructions and process data in the computer readable memory 1216, etc.

The computer readable memory 1216 may include computer program and/or operating instructions that the computer processor 1204 executes in order to implement one or more examples. The computer readable memory 1216 can store an operating system 1218 that provides computer program instructions for use by the computer processor 1204 in the general administration and operation of the computing system 1202. The computer readable memory 1216 can further include computer program instructions and other information for implementing aspects of the present disclosure. For example, in one example, the computer-readable memory 1216 may include a set of measurement instructions 1220 and a temperature calculator 1222 that, for example, implement the monitoring and calculation operations described elsewhere herein.

In some examples, multiple computing systems may communicate with each other via their respective network interfaces 1206 via a network, and can implement temperature acquisition and monitoring features independently (for example, each server may execute one or more separate instances of the processes or methods described herein), in parallel (for example, each system may execute a portion of one or more of the processes or methods described herein), etc. For example, a distributed computing environment may provide hosted capabilities for implementing the systems and methods described herein.

A person skilled in the art would understand that the devices, systems and methods described herein may be similar to the temperature monitor computing system 1202 as described herein. For example, the temperature acquisition devices 340 or 450 may include the components as described with respect to FIG. 12 above. For example, temperature measurement device 450 may include an on-board circuit the interfaces with a network and displays an output via a display screen located on the device. A person skilled in the art would understand how to configure each device to include, for example, wireless transmission capabilities by including a network chip and programming the device to communicate over an appropriate network protocol with one or more devices.

### Terminology

All of the methods and tasks described herein may be performed and fully automated by a computer system. The computer system may, in some cases, include multiple distinct computers or computing devices (including, for example, physical servers, workstations, storage arrays, cloud computing resources, and the like) that communicate and interoperate over a network to perform the described functions. Each such computing device typically includes a processor (or multiple processors) that executes program instructions or modules stored in a memory or other non-transitory computer-readable storage medium or device (including, for example solid state storage devices, disk drives, and the like). The various functions disclosed herein may be embodied in such program instructions, or may be implemented in application-specific circuitry (including, for example ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

The disclosed processes or methods may begin in response to an event, such as on a predetermined or dynamically determined schedule, on demand when initiated by a user or system administer, or in response to some other event. When the process or method is initiated, a set of executable program instructions stored on one or more non-transitory computer-readable media (including, for example hard drive, flash memory, removable media, and the like) may be loaded into memory of a server or other computing device. The executable instructions may then be executed by a hardware-based computer processor of the computing device. In some embodiments, the process or method or portions thereof may be implemented on multiple computing devices and/or multiple processors, serially or in parallel.

Depending on the embodiment, certain acts, events, or functions of any of the processes or algorithms described herein can be performed in a different sequence, can be added, merged, or left out altogether (including, for example not all described operations or events are necessary for the practice of the algorithm). Moreover, in certain embodiments, operations or events can be performed concurrently, for example, through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (including, for example ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (including, for example Windows XP, Windows Vista, Windows 7, Windows 8, Windows 10, Windows Server, and the like), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the scope of the disclosure. As can be recognized, certain embodiments described herein can be embodied within a form that does not provide all of the features and benefits set forth herein, as some features can be used or practiced separately from others. All changes which come within the meaning and range of equivalency of the claims are to be embraced within their scope.

1.-29. (canceled)

30. A method for estimating core body temperature, the method comprising:
receiving, using a temperature measurement device, a sample of air from a subject's lungs;
measuring temperature of the sample of air;
estimating a core body temperature measurement based at least in part on the measured temperature;
receiving supplemental physiological data from the subject;
refining the core body temperature measurement based at least in part on the supplemental physiological data; and
displaying a reading of the refined core body temperature measurement.

31. The method of claim 30, wherein the temperature measurement device comprises an intubation tube.

32. The method of claim 30, wherein the temperature measurement device comprises a hand-held device.

33. The method of claim 30, wherein the method further comprises:
receiving, from another temperature measurement device, a surface temperature measurement for the subject as measured over time; and
wherein the reading includes a plot of the refined core body temperature and surface temperature over time.

34. The method of claim 33, wherein the plot includes a heat map showing a duration of time that the subject was experiencing a particular refined core body temperature and surface temperature measurement relative to the duration of time the subject was experiencing a different refined core body temperature and surface temperature measurement.

35. The method of claim 30, wherein the supplemental physiological data includes one or more of: pulse oximetry data, respiration rate, blood pressure, perfusion index, surface temperature, circadian rhythm, or heart rate.

36. The method of claim 30, wherein the method further comprises:
signaling an alarm based at least in part on one or more of: the estimated core body temperature or the refined core body temperature.

37. A method for determining the condition status of a subject, the method comprising:
estimating, using a temperature measurement device, a core body temperature measurement for a subject;
receiving, from another temperature measurement device, a surface temperature measurement for the subject;
determining perfusion information of the subject; and
determining a trend for the subject's condition based at least in part on the core body temperature measurement, the surface temperature measurement, and the perfusion information of the subject.

38. The method of claim 37, wherein the method further comprises identifying a fever status based at least in part on the determined trend.

39. The method of claim 38, wherein the method further comprises classifying the fever status of a fever of the subject based at least in part on the determined trend.

40. The method of claim 37, wherein the method further comprises controlling application of a fever reducing device based at least in part on the determined trend.

41. The method of claim 40, wherein the fever reducing device comprises medication administered via an intravenous line.

42. The method of claim 37, wherein determining the trend is further based at least in part on a change in surface temperature, a change in core body temperature, and a change in perfusion index.

43. The method of claim 37, wherein determining the trend is further based at least in part on one or more of: pulse oximetry data, respiration rate, blood pressure, circadian rhythm, or heart rate.

44. The method of claim 37, further comprising displaying, via a user interface, a fever status classification along with a reading of the core body temperature.

45. A temperature acquisition system comprising:
a temperature measurement device; and
one or more computer hardware processors configured to execute program instructions to cause the temperature acquisition system to at least:
receive a first surface temperature measurement at a first location of a subject;
receive a second surface temperature measurement at a second location of the subject distinct from the first location;
determine a first perfusion index measurement at the first location of the subject;
receive a second perfusion index measurement at the second location of the subject; and
determine a condition of the subject based at least in part on one or more of: the first surface temperature measurement, second surface temperature measurement, the first perfusion index measurement, or the second perfusion index measurement.

46. The temperature acquisition system of claim 45, wherein the program instructions further cause the temperature acquisition system to:
determine a change in the first surface temperature measurement at the first location of the subject and a change in the second surface temperature measurement at the second location of the subject; and
determine a time delay difference between the change in the first surface temperature measurement at the first location and the change in the second surface temperature measurement at the second location.

47. The temperature acquisition system of claim 45, wherein the first surface temperature measurement and the second surface temperature measurement are received concurrently.

48. The temperature acquisition system of claim 45, wherein the condition of the subject indicates that the subject is experiencing one or more of: internal bleeding, compartment syndrome, or an infection.

49. The temperature acquisition system of claim 45, wherein an alarm is provided to a user based at least in part on the condition of the subject.
